(19) United States
(12) Patent Application Publication
(10) Pub. No.: US 2013/0037047 A1
(43) Pub. Date: Feb. 14, 2013

(54) Method for machine cleaning and disinfecting objects

(71) Applicant: Lothar Saiger, Duermentingen (DE)

(72) Inventor: Lothar Saiger, Duermentingen (DE)

(21) Appl. No.: 13/643,509

(22) PCT Filed: Apr. 26, 2011
(86) PCT No.: PCT/EP11/56595

§ 371 Date: Oct. 25, 2012

(30) Foreign Application Priority Data
Apr. 28, 2010 (DE) 10 2010 028 339.8

(51) Int. Cl. B08B 3/12

(52) U.S. Cl. 134/1

(57) ABSTRACT

The invention concerns a method for machine cleaning and disinfecting objects, in particular medical and/or dental instruments and/or work equipment. According to the invention, the objects are ultrasonically cleaned in water in a working area of a cleaning and disinfection system, wherein after cleaning of the objects the water is removed from the working area up to a defined residual volume, The residual volume of water remaining in the working area is subsequently converted into water vapour by means of a heating element arranged in the working area (18). The objects (40) are thermally disinfected in the working area by means of the water vapour, wherein a vapour atmosphere in the working area s subsequently removed from the working area for drying the objects.

The invention relates to a method for machine cleaning and disinfecting objects, in particular medical and/or dental instruments and/or work equipment.

Contaminated medical products and work equipment has to be cleaned, disinfected and optionally also sterilised before being reused on people or animals. The statutory requirements are contained in numerous hygiene laws.

Nowadays “washer machines” are used for this purpose. These machines have serious drawbacks: inadequate cleaning performance, unwashed areas, high costs, use of chemicals harmful to health, length of time required, resistances.

The invention addresses the problem of specifying a method for machine cleaning and disinfecting objects which permits a simpler, faster, more reliable and also more cost-effective cleaning and disinfection of objects.

That problem is solved according to the invention by a method having the features of independent claim 1.

Further advantages and advantageous embodiments of the subject matter of the invention are apparent from the description, the drawings and the claims. A significant advantage of the method according to the invention is that reproducible cleaning and disinfecting results can be achieved therewith (validation).

Contaminants and germs adhering to the objects are first of all efficiently detached from the objects by the ultrasound and suspended in the water, and in addition agglomerations of germs and agglutinations in the water are dispersed. This procedure fully replaces manual cleaning. The ultrasonic cleaning takes place even inside objects, if the objects have cavities.

Cleaning additives and substances having a disinfecting action can be added to improve efficiency.

According to the invention a defined residual volume (sufficient for disinfection) of the water used during the ultrasonic cleaning is evaporated in the working area after the ultrasonic cleaning.

The significant advantages according to the invention, compared with heating the entire ultrasonic bath, are that the smaller residual volume can be heated more rapidly and that temperatures higher than 100 degrees Celsius can be reached due to the evaporation. An external steam generator is unnecessary. The disinfection result can be optimised by heating the ultrasonic bath, the duration of the exposure to the steam and the temperature of the water vapour. After the cleaning and disinfection, the objects can be dried passively, by evaporation of the residual water on the surface of the objects, and actively, for example, by a fan.

According to the invention the objects can be irradiated with germicidal UV rays during the ultrasonic cleaning. This ensures that germs in the water and on the objects have already been depleted, i.e. killed, by the UV rays prior to steam disinfection of the objects. At the same time, the water permeable to UV rays is continuously disinfected by the UV radiation. Applying UV additionally offers the advantage of lack of bacterial resistance, The degree of cleaning and disinfection achieved with the combined ultrasonic cleaning and UV radiation, together with the subsequent steam disinfection, facilitates a synergistic disinfection effect.

According to a preferred development of the invention, the residual volume of water remaining in the working area is converted into water vapour by means of an electric heating element, in particular by means of a heating coil and/or by means of an infrared radiator. If a heating coil is used, the water can be converted into water vapour very swiftly and without technical effort,

In the case of the alternative or additional use of one or more infrared radiators, the objects, if they are no longer wet with the residual volume of water to be evaporated, can advantageously be brought to temperatures of more than 100° Celsius independently of the water being heated up. The thermal disinfection process can therefore start even more quickly. A fan can additionally assist the thermal disinfection by circulating the vapour atmosphere.

In order to ensure a reproducible degree of disinfection, the thermal disinfection of the objects is carried out over a relatively long period of time.

Water vapour having a temperature of from 100° to 140° Celcius is preferably used for thermal disinfection of the objects. Temperatures of approximately 134° Celcius and above can be used in particular in the case of thermo-resistant germs.

According to a development of the invention, saturated steam is preferably used in the thermal disinfection.

The disinfection and cleaning of the objects is further improved and accelerated as a result of the water vapour generated in the working area being fed to the objects (additionally) in the form of a steam jet by means of at least one nozzle. The objects can therefore be heated more quickly and more uniformly to a predetermined temperature and at the same time additionally be cleaned. The described cleaning effect corresponds to the mechanism of action of a steam jet cleaner. This procedure has the advantage that the selective directing of the water vapour counteracts the formation of air cushions, which could jeopardise a successful disinfection.

A pressure relief valve or an outlet opening preferably having an adjustable flow cross-section ensures that the pressure in the working area does not increase significantly uncontrollably beyond the atmospheric air pressure.

In a further development of the invention, the objects are irradiated with UV rays before the ultrasonic cleaning, during evaporation of the residual water in the working area and optionally also during the thermal disinfection.

For effective cleaning of objects having an externally accessible cavity, in particular dental and medical hand-pieces and angle pieces, according to the invention these are connected to a flushing device of the cleaning and disinfection system, the objects being flushed through with the (liquid) water during the ultrasonic cleaning and with the water vapour during the thermal disinfection. As a result, the contaminants and germs detached by ultrasound from inner surfaces of the objects can be flushed out of the objects more efficiently. At the same time it is ensured that the vapour comes immediately into direct contact with the inner surfaces of the objects, so that these too are rapidly and reliably disinfected.

After the cleaning, the objects, for example, the medical instruments, are heated, and the water in the cavities evaporates in situ and reliably and efficiently disinfects even cavities that are not readily accessible.

Independently of the heating of the residual water in the working area, a steam generator can be provided in the working area, separately from all apparatus components; the steam generator is able to generate additional steam and this steam can be emitted directly onto the objects to be disinfected or can be blown into cavities of the objects to be disinfected.

According to the invention the cleaning and disinfection parameters of the cleaning and disinfection system can be controlled by a control unit. The reproducibility of the cleaning and disinfection results is ensured by defined programme sequences.

According to the invention all (operating) parameters detected by the control unit can be electronically documented and can thus be used at any time to validate the cleaning and disinfection process.

The invention is explained in detail hereafter by means of an exemplary embodiment illustrated in the drawing.

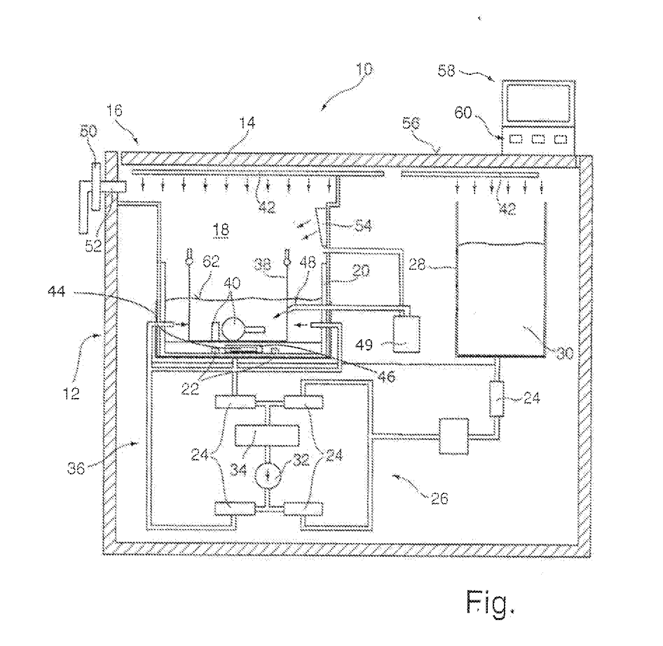

The single FIG. 1 shows a highly schematised section through a machine cleaning and disinfection system 10 according to the invention, for example, for dental instruments.

The cleaning and disinfection system 10 comprises a housing 12 impermeable to ultraviolet (UV) rays and infrared rays, having a housing cover 14 and a closable access opening 16 for introducing and removing objects to be cleaned and disinfected.

The housing 12 encloses a working area 18 with an ultrasound tank 20, which has two ultrasound sources 22. The ultrasound tank 20 is connected via a pipe work system 26 provided with controllable valves 24 to a storage container 28 for water 30. The water 30 stored in the storage container 28 can be conveyed by means of a pump 32 through the pipe work system 26 into the ultrasound tank 20. A heating device 34 associated with the pipe work system 26 enables the water 30 to be conveyed to the ultrasound tank 20 to be heated as needed.

The water 30 in the ultrasound tank 20 can be conducted in the pipe work system 26 along a closed circuit 36, thus enabling a defined circulation of the water 30 in the ultrasound tank 20. It is thus ensured that the water 30 flows in a defined manner over and through objects 40 positioned in the ultrasound tank 20 in an insert 38.

A UV radiator 42 for emitting ultraviolet rays is arranged above each of the ultrasound tank 20 and the storage container 28. The ultrasound tank 20 is made of reflective stainless steel.

The cleaning and disinfection system 10 further comprises a heating element 44 designed to evaporate water contained in the working area 18, and which in this particular case is embedded in a thermo-resistant body 46,

The water vapour generated in the working area 18 can be circulated and then fed in the form of a steam jet with a high steam flow rate via a nozzle 48 to the objects 40 to be cleaned. A fan 49 is used for circulating the water vapour.

To limit an atmospheric pressure prevailing in the working area 18 the housing 12 has an outlet 52 provided with an adjustable pressure relief valve 50; if needed, water vapour in the working area 18 is able to escape from the working area 18 via the outlet when a preset threshold pressure is reached.

The working area 18 furthermore comprises an infrared emitter 54, by means of which the working area 18 and the objects 40 located therein can be heated in a controlled manner.

The cleaning and disinfection system 10 comprises a control unit 58 arranged on an outer face 56 of the housing 12 and having an operator control panel 60.

All (operating) parameters detected by the control unit are electronically documented in a documentation centre, not shown more specifically, and can be used at any time to validate the cleaning and disinfection.

The method according to the invention for cleaning and disinfecting objects is explained in detail hereafter.

In a first step, the objects 40 to be processed, for example, medical instruments, are introduced via the access opening 16 of the housing 12 closable by the housing cover 14 into the ultrasound tank 20 of the working area 18 of the cleaning and disinfection system 10.

The access opening 16 is subsequently closed by means of the housing cover 14 and a cleaning programme defined in the control unit 58 is selected and started by way of the operator control panel 60 of the control unit 58.

As a result, the UV radiators 42 are activated. The first irradiation phase acts directly on the objects and via the reflectors also in regions that are inaccessible for direct irradiation. By means of the pump 32 and the pipe work system 26 water 30 is subsequently pumped into the ultrasound tank 20 until the water 30 has reached a filling level defined by the cleaning programme. The UV radiation emitted by the UV radiator 42 is reflected both at the water surface 62 and at the ultrasound tank 20, so that the objects 40 to be cleaned are comprehensively exposed to the UV radiation.

At the same time the ultrasound sources 22 are activated and ultrasonic cleaning of the objects 40 is commenced.

During the ultrasonic cleaning the objects 40 and all components disposed in the ultrasound tank 20 and wetted with water 30 are freed from contaminants by the ultrasound and are thus continuously cleaned. In this connection, larger conglomerates of contaminants and germs are split up into smaller particles. These particles are entrained as suspended matter by the water 30, which is circulated in the pipe work system 26 by means of the pump 24, wherein germs within the working area 18 are hit by the UV rays and killed.

When a defined ultrasonic cleaning time defined in the cleaning programme is reached, the water 30 located in the ultrasound tank 20 is removed via the pipe work system 26 by means of the pump 32 up to a remaining (defined) residual volume of water 30, i.e. in the present case is transported to the storage container 28 for re-use. An irradiation phase with UV radiation follows, during which the cleaned objects are further exposed to UV radiation.

At the same time, the electric heating element 44 is activated and the residual volume of water 30 remaining in the working area 18 is heated and evaporated to saturated water vapour,

When a predetermined pressure is exceeded, the water vapour in the working area 18 is expelled from the working area 18 within seconds via the outlet 52 fitted with the pressure relief valve 50. The UV irradiation of the ultrasound tank 20 and the objects 40 is continued throughout this steam disinfection of the objects 40.

When a residence time of the saturated vapour predetermined by the cleaning programme is reached, the steam disinfection is terminated and the vapour atmosphere in the working area 18 is replaced by filtered ambient air via a ventilation system, not more specifically shown. As a result, any residual moisture remaining in the working area 18 is dissipated and the cleaned and disinfected objects 40, preferably after they have cooled, are removed from the cleaning and disinfection system in a dry state, i.e. with no significant residual moisture, for subsequent, optionally immediate, use, for example, on a patient.

The invention relates to a method for the machine cleaning and disinfection of objects 40, in particular medical and/or dental instruments and/or work equipment. According to the invention, the objects 40 are ultrasonically cleaned in water 30 in a working area 18 of a cleaning and disinfection system 10, wherein after cleaning of the objects 40 the water 30 is removed from the working area 18 up to a defined residual volume. The volume of water 30 remaining in the working area 18 is subsequently converted into water vapour by means of a heating element 44 arranged in the working area 18. The objects 40 are thermally disinfected in the working area 18 by means of the water vapour, wherein a vapour atmosphere in the working area 18 is subsequently removed from the working area 18 for drying of the objects 40.

1. A method for machine cleaning and disinfecting objects, in particular medical and/or dental instruments and/or work equipment, in which the objects are ultrasonically cleaned in water in a working area of a cleaning and disinfection system, wherein after cleaning of the objects the water, is removed from the working area up to a defined residual volume, wherein
the residual volume of water remaining in the working area subsequently converted into water vapour by means of a heating element arranged in the working area, wherein the objects in the working area are thermally disinfected by means of the water vapour and wherein subsequent to the vapour disinfection of the objects a vapour atmosphere in the working area is removed from the working area.

2. A method according to claim 1, wherein the objects are irradiated during the ultrasonic cleaning with germicidal UV rays.

3. A method according to claim 1, wherein the water is converted into water vapour by means of an electric heating element, in particular by means of a heating coil and/or by means of an infrared emitter.

4. A method according to claim 1 wherein the thermal disinfection of the objects is carried out over a period of at least 3 minutes.

5. A method according to claim 1 wherein the water vapour has a temperature of 100° Celcius to 140° Celcius, preferably 100° to 125° Celcius.

6. A method according to claim 1 wherein the water vapour is saturated steam.

7. A method according to claim 1 wherein the water vapour is supplied by means of at least one nozzle (48) in the form of a vapour jet directed onto the objects.

8. A method according to claim 1 wherein a pressure developing in the working area as a result of the vapour atmosphere is limited by a. preferably adjustable pressure relief valve or by an outlet opening, preferably having an adjustable flow cross-section, at the working area.

9. A method according to claim 1 wherein the working area and the objects are heated, preferably by irradiation with infrared rays, before the vapour disinfection to a temperature of above 100° Celcius.

10. A method according to claim 1 wherein the objects are irradiated with the UV rays before the ultrasonic cleaning, in particular before the objects are wetted with the water.

11. A method according to claim 1 wherein the objects have an externally accessible cavity, in particular dental and medical hand pieces and angle pieces are connected to a flushing device, wherein by means of the flushing device the objects are flushed through with the water during the ultrasonic cleaning of the objects and with the water vapour during the thermal disinfection.

12. A method according to claim 1 wherein the cleaning and disinfection parameters, in particular a particular residence time of the ultrasound and/or of the vapour are controlled by a control unit, wherein the individual parameters are freely preset via an operator control element associated with the control unit and/or wherein the parameters are preset by calling up cleaning programme defined in the control unit, preferably specifically designed for the cleaning of objects, and at least a selection of the parameters is documented.
